(19) United States
(12) Patent Application Publication
(10) Pub. No.: US 2007/0092550 A1
(43) Pub. Date: Apr. 26, 2007

(54) Methods and compositions for growing corneal endothelial and related cells on biopolymers and creation of artifical corneal transplants

(75) Inventor: Ge Ming Lui, Honolulu, HI (US)

Correspondence Address: JACOBSON HOLMAN PLLC, 400 SEVENTH STREET N.W., SUITE 600, WASHINGTON, DC, 20004, US

(73) Assignee: CELLULAR BIOENGINEERING, INC., Honolulu, HI (US)

(21) Appl. No.: 10/575,246

(22) PCT Filed: Oct. 7, 2004
(86) PCT No.: PCT/US04/32934

§ 371 Date: Dec. 11, 2006

Related U.S. Application Data
(60) Provisional application No. 60/510,359, filed on Oct. 10, 2003.
(60) Provisional application No. 60/510,349, filed on Oct. 10, 2003.
(60) Provisional application No. 60/510,350, filed on Oct. 10, 2003.

(51) Int. Cl. A61K 35/32; A61F 2/02

(52) U.S. Cl. 424/427; 424/93.7

(57) ABSTRACT

This invention discloses methods to attach and grow a monolayer of cultured human corneal endothelial cells onto the endothelial side of the stroma synthesized from biopolymer to generate a more bio-equivalent artificial cornea. The approaches will include the use of attachment and growth promoting agents such as fibronectin, laminin, RGDS, collagen type IV, bFGF conjugated with polycarbophil, and EGF conjugated with polycarbophil. The patent also describes a method to create a self-sustaining polymer containing adhesive molecules and growth factors to support the attachment and proliferation of cultured human corneal endothelial cells for corneal transplantation either as a half-thickness device or full-thickness button replacement. An approach for the implantation of cultured retinal pigment epithelial (RPE) cells into the sub-retinal space for treatment of age-related macular degeneration (ARMD) is disclosed in this invention. This method will enable the delivery of the transplanted RPE in a sheet of monolayer cells and will be better suited to perform their physiological function.

This patent application claims priority to U.S. patent application Ser. Nos. 60/510,359 filed Oct. 10, 2003; 60/510,350 filed Oct. 10, 2003; and 60/510,349 filed Oct. 10, 2003; and are all incorporated by reference herein as if set forth in its entirety.

### BACKGROUND OF THE INVENTION

1. Field of Invention

This patent describes improved methods of dissecting, seeding and subsequent propagation of pure culture of human corneal endothelial and retinal pigment epithelial cells on extracellular matrices, and the compositions and methods of making artificial corneal transplants.

2. Description of Prior Art

For various reasons, the corneal portions of eyes may need to be surgically repaired or replaced. For example, the cornea may become scratched or scarred or otherwise physically damaged, greatly hindering sight. The cornea is also subject to the effects of various degenerative diseases, mandating replacement if the patient is to have normal or even near normal vision.

The cornea of the human eye is a specialized structure made up of substantially parallel relatively compacted layers of tissue. The outermost or most superficial layer of the cornea is the epithelial layer. This is a protective layer of tissue which regenerates if injured. Moving inwardly in the eye is the base surface of the epithelial layer known as Bowman's membrane. Immediately adjacent the Bowman's membrane is the stroma of the cornea, which is an extra-cellular collagen architectural matrix with scattered keratocytic cells. The stroma layer is bounded at its deepest level by a cuticular, a cellular membrane, referred to as Descemet's membrane, which is followed by a monolayer of single cell thickness of specialized endothelial cells which forms the posterior surface of the cornea. The endothelial layer does not regenerate and when it is diseased, scratched or otherwise injured, it must be replaced.

In some animal species including human, the corneal endothelium does not normally replicate in vivo to replace cells lost due to injury or aging (Murphy C, et al., Invest. Ophthalmology Vis. Sci. 1984; 25:312-322; Laing R A, et al., Exp. Eye Res. 1976; 22:587-594). However, human corneal cells can be cultured in vitro with a growth factor-enriched, fetal calf serum-containing medium under normal tissue culture conditions (Baum J L, et al., Arch. Ophthalmol. 97:1136-1140, 1979; Engelmann K, et al., Invest. Ophthalmol. Vis. Sci. 29:1656-1662, 1998; Engelmann K, and Friedl P; In Vitro Cell Develop. Biol. 25:1065-1072, 1989). If the cultured cells can be utilized to replace the loss of corneal endothelial cells it will greatly enhance the donor pool of human corneas. This is important as one may be able to augment the donor corneas currently rejected for transplantation procedures due to inadequate endothelial cell counts (Gospodarowicz D, et al., Proc. Natl. Acad. Sci. (USA) 76:464-468, 1979; Gospodarowicz D, et al., Arch. Ophthalmol. 97:2163-2169, 1979). This pool [the ones rejected due to low cell counts?] of corneas-makes up to 30% of the total donated corneas annually (National Eye Institute: Summary report on the cornea task force. Invest Ophthalmol Vis Sci 12:391-397, 1973). Furthermore, a method to culture human corneal endothelial cells from a low initial density, and the ability to reseed the cells grown in vitro onto denuded corneal buttons, will enable the use of the recipient's own undamaged stroma for allo-cell and auto-stroma type of transplantation (Insler M S, and Lopez J G, Cornea. 10:136-148, 1991).

Tissue culture techniques are being successfully used in developing tissue and organ equivalents. The basis for these techniques involve collagen matrix structures, which are capable of being remodeled into functional tissue and organs by employing the right combination of living cells, nutrients, and culturing conditions. Tissue equivalents have been described extensively in many patents, including U.S. Pat. Nos. 4,485,096; 4,485,097; 4,539,716; 4,546,500; 4,604,346; 4,837,379; and 5,827,641, all of which are incorporated herein by reference. One successful application of the tissue equivalent is the living skin equivalent, which has morphology similar to actual human skin. The living skin equivalent is composed of two layers: the upper portion is made of differentiated and stratified human epidermal keratinocytes that cover a thicker, lower layer of human dermal fibroblasts in a collagen matrix (Bell, et al., J. of Biochemical Engineering, 113:113-19 (1991)).

Studies have been done on culturing corneal epithelial and endothelial cells (Xie, et al., In Vitro Cell. Develop. Biol., 25:20-22 (1989) and Simmons, et al., Tox. App. Pharmacol., 88:13-23 (1987)).

Due to chronic worldwide shortage of donor human corneas, there has been ongoing interest in the generation of artificial corneal stroma for transplantation in patients with both endothelial and epithelial diseases of the cornea, as well as traumatic rupture of the cornea in accidents requiring total corneal replacement.

Currently, most of the attempts to generate a substitute corneal stroma rely on the use of polymer gels, either from natural sources or synthetic combination by cross-linking the protein moieties in the polymer. Since most of the polymer gels contain up to 80% of the total volume in aqueous phase, they will become swollen if the artificial corneal stroma is placed in contact with aqueous fluid. In this instance a transplanted artificial corneal stroma will be constantly subjected to the aqueous environment of the exterior chamber. The subsequent swelling of the polymer gel will cause haziness in the polymer gel as well as visual distortion due to increased thickness of the artificial stroma. It is therefore desirable to place a layer of cultured human endothelial cells on the inside of the artificial stroma to act as a barrier for fluid penetration and also to keep the stroma at the right thickness by constantly pumping fluid out in a basal to apical direction, this keeping the artificial stroma thin and maintain a high degree of clarity.

It is advantageous when creating an artificial stroma for corneal transplantation, to include agents which will induce and sustain cell attachment and proliferation into the biopolymer during its synthesis. An artificial cornea that can support three distinct cell types, namely, the corneal epithelial cells on the convex side, the keratocytes in the interior, and the corneal endothelial cells on the on the concave side, can act as a corneal equivalent much more closely than a mere device. The corneal endothelial layer acts as a fluid barrier which pumps fluid outwards constantly. The keratocytes to grow out from the wound and anchor the transplanted artificial cornea in place, the corneal substitute can achieve a state of relative dehydration maintained by the normal intact cornea that enables it to remain transparent (detergence) and stability after the transplant procedure.

In addition to corneal trauma age-related macular degeneration occurs in humans naturally as an aging disease (Gartner S, and Henkind P., Br. J. Ophthalmol. 1981 January; 65(1):23-8; J. Marshall et al., Br. J. Ophthalmol. 1979, Vol 63, 181-187). The retinal pigment epithelium (RPE) is suggested to be heavily involved in these degenerative diseases due to its loss of biological and physiological functions as a result of high stress caused by constant cellular activities such as phagocytosis of rods' outer segment and cumulative exposure to toxic factors (Dorey C K., et al., Invest. Ophthalmol. Vis. Sci. 1989 August; 30(8):1691-9; Hogan M J, Trans. Am. Acad. Ophthal.-mol Otolaryngol 1972; 7:64-80). RPE cell transplantation has been proposed as a possible treatment for human degenerative macular and peripheral retinal diseases (Li, L. and Turner, J E., Exper. Eye Res. 47:911 (1988); Lane, C., et al., Eye. 1989; 3 (Pt 1):27-32). The ramifications of these proposals create the need for surgical venues for delivery of human RPE cells into the sub-retinal space during the cell transplantation procedure. Direct injection of APE cells suspensions into the sub-retinal space as a method of RPE cell transplantation fells short of the expected clinical outcome due to the aggregation of the injected cells to form clumps instead of settling down as a monolayer, a necessary condition for them to function properly (Gouras P G., et al., Curr. Eye Res. 1985; 4: 253-265; Lopez R., et al., Invest. Ophthalmol. Vis. Sci. 1987; 28: 1131-1137). To transplant the cultured RPE cells as a monolayer resting on a sheet of biodegradable polymer membrane will solve the problem.

Until the advent of the present invention, prior art methods of culturing human corneal endothelial cells (HCEC) encountered problems such as the fact that HCEC cells could only be seeded at high cell density (2000-5000 cells/square mm) therefore limiting the possibility to start a primary culture from small specimen, and that HCEC cells could not be passaged continuously at low seeding density (50-100 cells/square mm) which limits the ability to expand the HCEC stock for storage and future use.

### SUMMARY OF THE INVENTION

The present invention provides a method for modifying a biopolymer surface to enhance cultured corneal endothelial cell attachment a subsequent growth on the biopolymer. In particular, the cultured cells will be able to remain attached to the biopolymer surface and perform their physiological functions such as forming tight junctions to prevent fluids from entering into the biopolymer to cause unwanted swelling, as well as to exhibit active Na/K pump activity in basal to apical direction to remove excess fluid from the biopolymer so that the detergence and clarity of the substitute corneal stroma biopolymer) will be maintained.

The approach of the present invention involves the use of attachment proteins such as fibronectin, laminin, RGDS, collagen type IV, bFGF conjugated with polycarbophil, and EGF conjugated with polycarbophil. Polycarbophil is a lightly cross-linked polymer. The cross linking agent is divinyl glycol. Polycarbophil is also a weak poly-acid containing multiple carboxyl radicals which is the source of its negative charges. These acid radicals permit hydrogen bonding with the cell surface. Polycarbophil shares with mucin the ability to adsorb 40 to 60 times its weight in water and is used commonly as an over-the-counter laxative (Equalactin, Konsyl Fiber, Mitrolan, Polycarb)(Park H, et al., J. Control Release 1985; 2:47-57). Polycarbophil is a very large molecule and therefore is not absorbed. It is also non-immunogenic, even in the laboratory it has not been possible to grow antibodies to the polymer.

In one embodiment of the present invention there is disclosed a self-sustaining polymer which embeds or has incorporated within the biopolymer during it's synthesis, an attachment mixture comprising of one or more of the following: fibronectin, laminin, RGDS, bFGF conjugated with polycarbophil, EGF conjugated with polycarbophil, and heparin sulfate. The biopolymer can be molded into any desired shape, with the shape of a cornea being preferred, and cultured human corneal endothelial cells will be seeded onto the concave surface and allowed to proliferate until confluent.

It is also contemplated that the present invention will disclose a self-sustaining biopolymer which can also be molded into half the thickness of the normal human cornea and covered with cultured human corneal endothelial cells for half-thickness transplantation in a process called Deep Lamellar Endothelial Keratoplasty (DLEK) Terry, M. A., Eye. 2003 November; 17(8):982-8; Loewenstein A, and Lazar M., Br. J. Ophthalmol. 1993; 77:538).

In another embodiment, the self-sustaining biopolymer can be molded into the shape of a cornea either in full or half-thickness and cultured human-corneal endothelial cells will be seeded on the concave side of the artificial stroma, after an 11 mm diameter button has been punched out by trephination.

It is also an object of the present invention to provide a biopolymer surface with a diamond like coating of carbon and treating said coated surface with an attachment mixture that creates a biopolymer surface suitable for growing corneal endothelial cells in vitro.

Another embodiment of the present invention involves the use of a thin (10-100 micron in thickness) biopolymer sheet as a carrier for retinal pigment epithelial (RPE) cell transplantation into the sub-retinal space of the eye for the treatment of age-related macular degeneration (ARMD). Alternatively, a thin sheet of biodegradable polymer can be used as the carrier of the cultured RPE cells for the transplantation procedure. The advantage of using the biodegradable system is that RPE, cells can get into contact with the Bruch's membrane and the underlying vasculature system soon after the polymer is degraded and to perform its transport and phagocytosis, function sooner.

These and other objects of the invention, as well as many of the attendant advantages thereof, will become more readily apparent when reference is made to the following detailed description of the preferred embodiments.

### BRIEF DESCRIPTION OF THE DRAWINGS

FIG. 1 shows generation curves for long term serial propagation of cultured human endothelial cells on different substrates.

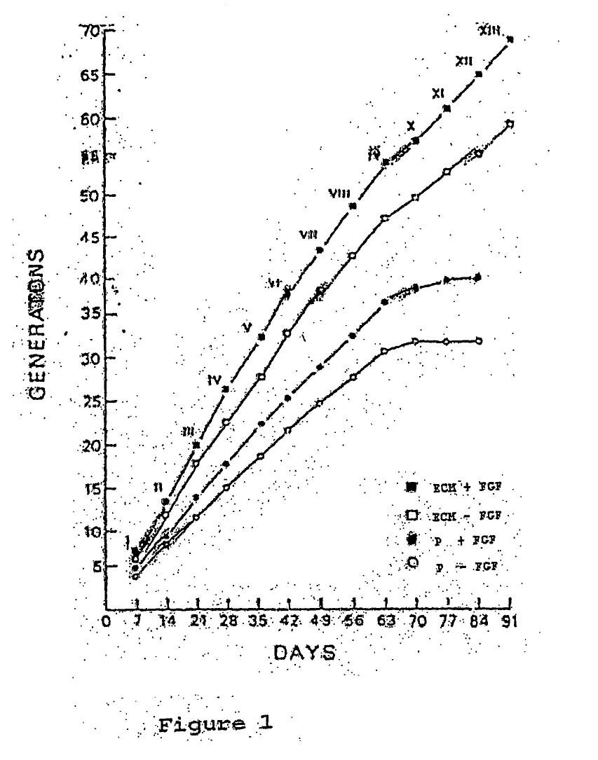

FIG. 2 illustrates the effects of various attachment factors on the proliferation of cultured human corneal endothelial cells in the presence or absence of bFGF.

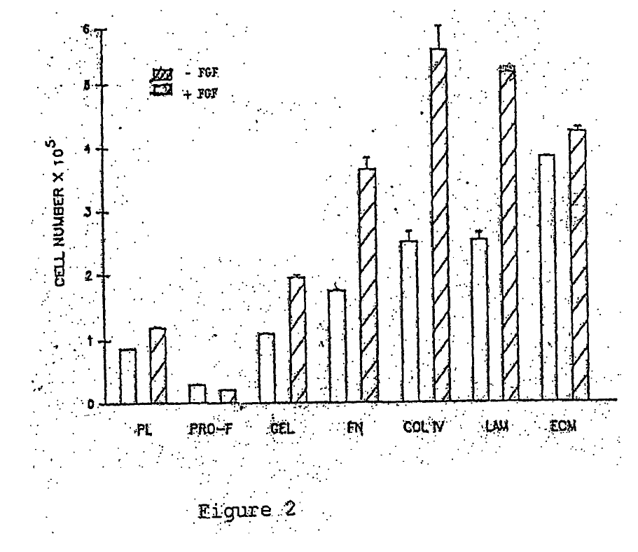

FIG. 3 is a time curve of attachment of cultured human corneal endothelial cells onto the denuded human corneal buttons coated with attachment agents.

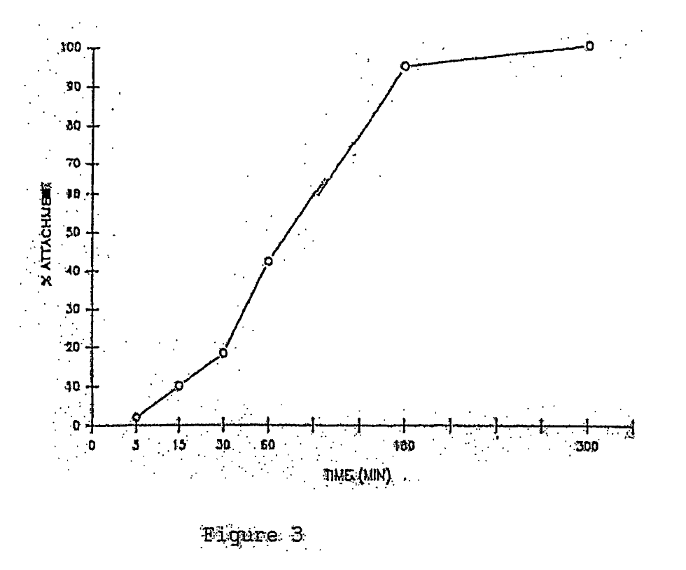

### DETAILED DESCRIPTION AND PREFERRED EMBODIMENTS

In describing a preferred embodiment of the invention specific terminology will be resorted to for the sake of clarity. However, the invention is not intended to be limited to the specific terms so selected, and it is to be understood that each specific term includes all technical equivalents which operate in a similar manner to accomplish a similar purpose.

Previous studies have demonstrated that human corneal endothelial cells (HCEC) can be grown on polymer surfaces (T. Mimura et al., 2004 Invest. Ophthal. Vis. Sci. Vol. 45. No. 9-2992-2997; F. Li et al., 2003; Proc. Nat. Acad. Sci. USA Vol 100. 15346-15351). However, these cells can remain attached to the polymer beads for 12-14 weeks at the maximum M. S. Insler and J. G. Lopez, 1989 Curr. Eye Res. Vol. 9:23-30).

The present invention describes methods that modify the endothelial side of an artificial stroma. This is made possible by the long term attachment of the cultured HCEC and their ability to perform vital biological functions to maintain the integrity and detergence of the artificial cornea. To this end, the present invention discloses a predefined mixture of attachment proteins and growth factors (attachment mixture), namely, fibronectin at concentrations ranging from 0.1 μg to 500 μg/ml in PBS, laminin at concentrations ranging from 0.1 μg to 500 μg/ml in PBS, RGDS at concentrations ranging from 0.1 μg to 200 μg/ml in PBS, collagen type IV at concentrations ranging from 1 μg to 1000 μg/ml in 0.01 M acetic acid, collagen type I at concentrations ranging from 1 μg to 1000 μg/ml in 0.01 M acetic acid, bFGF at concentrations ranging from 1 ng to 500 ng/ml in PBS conjugated with polycarbophil (at 0.01 μg/ml), and ESF in concentrations ranging from 1 ng to 500 ng/ml in PBS conjugated with polycarbophil.

The predefined attachment mixture will be added to the concave side of a polymer-gel which is molded into the shape of a cornea. The polymer-gel is then incubated at 4° C. for a period of time ranging from 20-minutes to 24 hours. Afterwards the residual attachment mixture is removed and the cornea is ready for seeding of cultured corneal endothelial cells. In an alternative embodiment, a native extracellular matrix derived from cultured bovine endothelial cells can be deposited directly onto the polymer.

Corneal endothelial cells from bovine origin are seeded on the endothelial side of the cornea-shaped polymer gel. The device will be left concave side up in a 35 mm culture dish with the seeded cells in the well space and incubated for 2 hours at 37° C. in a 10% CO₂ incubator. Approximately 2 ml of culture medium (supplemented with 10% calf serum, 5%, fetal calf serum, and 2% w/v dextran (MV 40,000) will be added to totally submerge the artificial cornea. The bovine endothelial cells are allowed to grow to confluency for seven days. Then the endothelial cell layer in treated with 20 mM ammonium hydroxide solution in distilled water for 5 minutes, rinsed 10 times with PBS, and the artificial cornea stroma is ready for coating with cultured human corneas endothelial cells. In another embodiment, the artificial cornea stroma can be coated with diamond-like carbon (DLC), using a plasma gun for depositing a thin layer of carbon onto the cornea shaped polymer in a vacuum environment.

In one alternative embodiment, the corneal endothelial cells used to form the endothelial layer can be derived from a variety of mammalian sources. Non-transformed corneal endothelial cells derived from sheep, rabbit, and cows have been used. Mouse corneal-endothelial cells have been transformed with large T antigen of SV40. (Muragaki, Y., et al., Eur. J. Biochem. 207(3):895-902 (1992). Non-human cell types which can be used also include transformed mouse corneal endothelial cell lines, or normal corneal endothelial cells derived from sheep or rabbit. The normal rabbit endothelial cells can be derived from enzymatically dissociated corneal endothelium or from explants of cornea and are, serially cultivated in MSBM medium (Johnson, W. E. et al., In Vitro. Cell. Dev. Biol. 28A:429-435 (1992)) modified by the addition of 50 μg/mL heparin and 0.4 μg/mL heparin binding growth factor-1 (MSBME).

In yet another embodiment, endothelial cells from a non-corneal origin may also be used in this invention. The non-corneal origin endothelial cells that have also been used in this invention include ovine and canine vascular and human umbilical vein endothelial cells. The endothelial cells may be transformed with a recombinant retrovirus containing the large T antigen of SV40 (Muragaki, et al., 1992, supra). Transformed cells continue to grow in the corneal equivalent and form mounds on top of the a cellular layer due to their lack of contact inhibition. Non-transformed cells will form a monolayer underlying the stromal cell-collagen layer. Alternatively, normal endothelial cells may be transfected as above, but with the addition of a recombinant construct that expresses a heat sensitive gene. These transformed cells will grow in continuous culture under reduced temperature. After establishment of a confluent endothelial cell layer, the temperature can be raised to deactivate the transforming gene, allowing the cells to resume their normal regulation and exhibit, contact inhibition, to form an endothelial cell monolayer similar to the non-transformed cells. Most peptides are heat sensitive (with the exception of heat shock proteins) so that there is a wide choice of peptides that can be deactivated by raising culturing temperature. Transformation in this way also facilitates the use of hard to obtain and cultivate cell types such as human corneal endothelial cells.

The self-sustaining polymer of the present invention will be generated by embedding, or incorporating into the biopolymer during its synthesis, attachment and/or growth promoting reagents comprising of one or more of the following: fibronectin at concentrations ranging from 0.1 μg to 500 μg/ml of polymer gel, laminin at concentrations ranging from 0.1 μg to 500 μg/ml of polymer gel, RGDS at concentrations ranging from 0.1 μg to 100 μg/ml of polymer gel, bFGF conjugated with polycarbophil at concentrations ranging from 1 ng to 500 ng/ml of polymer gel, EGF conjugated with polycarbophil in concentrations ranging from 10 ng to 1000 ng/ml of polymer gel, and heparin sulfate at concentrations ranging from 1 μg to 500 μg/ml of polymer gel. This enriched biopolymer is then molded into the shape of a cornea wither as a full thickness corneal substitute (the normal thickness of a human cornea) or a half thickness corneal substitute (up to half the thickness of a normal human cornea). Cultured human corneal endothelial cells will be seeded at low density (about 2000 to 150,000 cells/ml, preferably-20,000 cells/ml) onto the concave side of the artificial stroma and the culture will be grown from seven to ten days at 37° C. in a 10% CO₂ incubator.

When the corneal endothelial cells are confluent, as determined be observation under an inverted microscope, the cornea substitute will be rinsed three times with PBS and is now ready for transplantation.

In another embodiment, the artificial stroma, either in full thickness or half thickness form, can be seeded with a confluent layer of cultured human corneal endothelial cells by seeding the cells at the saturation density (about 0.5×10⁵ to 1×10⁷ cells/ml, preferably 10⁶ cells/ml) onto a button which is punched with an 11 mm trephine. A 200 μl aliquot of the cells will be added to the button and the sample will be incubated at 37° C. in 10% CO₂ for 2 hours to 24 hours. The corneal substitute will be rinsed three times with PBS and is ready for corneal transplantation.

To grow retinal pigment epithelial cells, a biocompatible biopolymer, whose composition and synthesis are known to individuals who are familiar with the skill of the trade, is molded into a thin sheet of uniform thickness between 1 and 1000 microns, preferably between 10 and 100 microns. RPE cells are grown onto the membrane to confluence or coated onto the membrane at high seeding density to cover over 95% of the membrane's surface. This RPE coated polymer sheet will serve as a carrier system for placement into the back of the eye.

To perform this procedure according to the present invention, the RPE coated sheet will be cut into desirable size sufficient to cover the damaged-RPE area on the Bruch's membrane. The piece will then be placed RPE cells up and aspirated into a cannula. The sheet will fold up with the RPE, cells located on the inside. To prepare the surgical site for the implantation in the sub-retinal space, an air bubble is injected into the sub-retinal space where the host RPE cell damage has been identified. This area will be cleaned up by aspirating the existing damaged RPE cells with a suction needle. The space is rinsed with balanced salt solution (BSS) once, and the sheet of folded RPE coated polymer will be deposited into place. The air bubble will be aspirated to return the retina to the normal form, thus holding the RPE sheet in place.

In another embodiment, the carrier sheet can be synthesized with a biodegradable polymer whose composition and synthesis is commonly known to individual familiar with the skill of the trade. Cultured RPE cells will be grown to confluence or deposited at high seeding density to cover the entire surface of the polymer sheet. Then the sheet is cut into the desired size and implanted in to the sub-retinal space of the eye as previously described.

With regard to the present invention, the biopolymer or the biodegradable form of a biopolymer, can be embedded with or incorporated into during the synthesis process an attachment reagent comprising of one or more of the following: fibronectin, laminin, RGDS, collagen type IV, bFGF conjugated with polycarbophil, and EGF conjugated with polycarbophil, and heparin sulfate. Cultured RPE cells can them be grown on such a polymer sheet until confluence, or be deposited at high seeding density to cover the entire surface. The sheet with RPE cells on it is then cut to the desired dimensions and implanted in the sub-retinal space as previously described.

### EXAMPLE 1

### Non-Enzymatic Dissection of Primary Human Corneal Endothelial Cells

The corneal rims from human donors (after the central portion has been removed for transplantation) or whole donor corneas will be rinsed in a large volume (50 ml) of phosphate buffered saline (PBS). They will then be placed in endothelial side up on a holder. The trabecular meshwork and remnants of iris will be removed carefully by micro-dissection. By using sharp pointed jeweler's forceps, the endothelial cell layers and the Descemet's membrane will be peeled off very carefully with great care taken not to include any underlying stromal tissue. This step can be confirmed by viewing the dissected Descemet's membrane under an inverted microscope to make sure it only carries the corneal endothelial cells on one side and nothing on the other side. The piece of tissue will be placed onto an ECM coated 35 mm tissue culture dish or similarly suitable container, filled with approximately 0.5 ml of culture medium (DME-H16 with 15% fetal calf serum enriched with b-FGF at 250 ng/ml). The dish will be incubated at 37° C. in a 10% CO₂-incubator for 24 hours, and then another 1 ml of culture medium will be added. The sample will be incubated without disturbance for about 7 days to see if a colony of corneal endothelial cells migrates outwards from the tissue sample, at which time (7 to 14 days after the sample is placed in culture) the medium is changed every other day until the cell count reaches 200-500 cells.

### EXAMPLE 2

### Culture of Human Corneal Endothelial Cells at High Split Ratio

When the primary cell count from the tissue sample outgrowth reaches a number of 200 to 500, the cells will be released from the dish with STV solution (0.05% trypsin, 0.02% EDTA in normal saline). The STV solution will be removed when the cells round up but are still attached to the culture dish. No centrifugation step is necessary since the remaining STV will be inactivated by the growth media containing 15% fetal calf serum. The corneal cells will be placed onto a 60-mm ECM-coated dish (about 500 cells per dish). The medium will be changed every other day and b-FGF at a concentration of 250 ng/ml will be added at the time of medium change. At confluence (about 7 to 10 days after plating), the cells will be passaged again at the same split ratio (1:16 to 1:64) or will be frozen in 10% DMSO, 15% FCS at a density of 10⁶ cells/ml per ampoule and stored in liquid nitrogen for future use. The passaging can be carried out for up to 8 times without loss of cell functions or morphological integrity.

Freezing of HCEC Stock.

For each of the 5 ml of HCEC collected, 0.5 ml of DMSO was added to the cell suspension. Each 1.1 ml of the mixture was aliquoted into a 1.5 ml cryopreservation tube to yield an approximate 1 million cells per vial final concentration. The vials were then put into a Styrofoam box and let stand in a −80° C. freezer for 24 hours. After 1 day, the ampoules were transfer into liquid nitrogen for long term storage.

### EXAMPLE 3

### Denudation of Corneal Button

Human donor corneal buttons are obtained from the Eye Bank. These corneal buttons are deemed unsuitable for transplantation-due to inadequate endothelial cell counts, but otherwise are healthy and disease free and obtained under eye banking guidelines.

The corneal button will be placed endothelial side up in a holder, and rinsed three times with PBS. Then a solution of ammonium hydroxide at a concentration ranging from 10 mM to 2200 mM will be added carefully into the corneal button without spilling over the top. The cornea will be kept at temperatures of about 10° C. to 25° C. for a period of 5 minutes up to 2 hours. Then the ammonium hydroxide will be removed, and the inside of the cornea button rinsed approximately 10 times with PBS. A cotton swab will be slid gently across the endothelial surface to remove any residual cell skeletons or debris. The corneal button is rinsed again three times with PBS, punched with an 11 mm trephine, and is then ready for coating with cultured human corneal endothelial cells.

Alternatively, the native corneal endothelium can be removed by adding Triton-X100 at a concentration of 0.5 to 5% in distilled water kept at 10° C. for a period ranging from 5 minutes to 2′ hours, and then processed as previously described. Furthermore, the corneal endothelium can be treated with distilled water for a period of 20 minutes to 2 hours at a temperature ranging from 4° C. to 25° C. Then the cotton swab will be slid gently across the endothelial surface to remove the cell cytoskeleton and debris. The cornea will then be processed with an 11 mm trephination.

### EXAMPLE 4

### Treatment of Denuded Corneas with Attachment Proteins and Growth Factors

After trephination, the denuded cornea button will be placed endothelial side up again in a holder. A solution of attachment proteins (attachment mixture) containing fibronectin at a concentration ranging from 10 μg to 500 μg/ml in PBS, laminin (10 μg to 500 μg/ml in PBS), RGDS (1 μg to 100 μg/ml in PBS), collagen type IV (10 μg to 1000 μg in 0.1 M acetic acid), b-FGF (1 to 500 ng/ml in PBS), EGF (1 ng to 500 ng/ml in PBS) will be added carefully onto the denuded cornea button. The specimen is allowed to incubate at 4° C. for a time ranging from 5 minutes to 2 hours, at the end of which the cocktail will be removed and the cornea rinsed 3 times with PBS.

### EXAMPLE 5

### Coating the Polymer with a Mixture of Attachment Agents and Growth Factors with High Density Cell Seeding

A biopolymer or polymer gel that satisfies the characteristics of an artificial stroma is molded into the shape of a cornea. This artificial stroma is placed concave side up and wetted with PBS. About 0.5-0.8 ml aliquot of the attachment mixture (containing fibronectin at concentrations ranging from 0.1 μg to 500 μg/ml in PBS, laminin at concentrations ranging from 0.1 μg to 500 μg/ml in PBS, RGDS at concentrations ranging from 0.1 μg to 200 μg/ml in PBS, collagen type IV at concentrations ranging from 1 μg to 1000 μg/ml in 0.01 M acetic acid, collagen type I at concentrations ranging from 1 μg to 1000 μg/ml in 0.01 M acetic acid, bFGF at concentrations ranging from 1 ng to 500 ng/ml in PBS conjugated with polycarbophil (at 0.01 μg/ml), and ESF in concentrations ranging from 1 ng to 500 ng/ml in PBS conjugated with polycarbophil) will be instilled into the concave surface of the cornea shaped polymer, and then the sample is incubated at 4° C. to 25° C. for a period of time, ranging from 10 minutes to 2 hours. The attachment mixture is removed; the artificial polymer corneal stroma is rinsed three times with PBS, and is ready for seeding of cultured human corneal endothelial cells. The cultured corneal endothelial cells are detached from the dish with STV solution (0.05% trypsin, 0.02% EDTA in normal saline). The endothelial cells are centrifuged at 2000 rpm for 5 minutes, and the cell pellet will be resuspended in 1 ml of DME-H16 culture medium supplemented with fetal calf serum at concentrations ranging from 0.1% to 5%. The cell-count will be determined with a Coulter Particle Counter and adjusted to about 10⁶ cells per ml. The artificial cornea is then punched with an 11 mm trephine and an aliquot of 200 ml (containing between 2000 and 2×10⁶ cells, preferably between 150,000 to 250,000 cells) will be seeded onto the cornea shaped stroma to cover 95% of the surface area.

The artificial cornea is incubated for 20 minutes to 24 hours prior to using for transplantation. A layer of 1% sodium hyaluronate (Healon® Advanced Medical Optics, Santa Ana, Calif.) of about 0.2-0.5 ml, is overlaid onto the cell layer to act as a protectant.

### EXAMPLE 6

### Coating the Biopolymers with Attachment Reagents and Growth Factors for Seeding of Sparse Density of Cultured Human Corneal Endothelial Cells

In another embodiment, the biopolymer is molded into the shape of a cornea. A sufficient quantity of attachment mixture is added to coat the concave surface of the artificial stroma, as previously described in Example 5. At the end of the incubation at 4° C., the attachment mixture is removed and the polymer cornea is rinsed three times with PBS. The polymer cornea is then punched with an 11 mm trephine while remaining hydrated with PBS in a 35 mm tissue culture dish. Cultured human corneal endothelial cells are detached from the culture dish as previously described. The endothelial cells will be spun down at 2000 rpm, resuspended in 5 ml of culture medium supplemented with 15% fetal calf serum. The cell quantity is determined with a Coulter Particle Counter and the cell density will be adjusted to about 100,000 cells per ml. An aliquot of about 100 μl of the cell suspension containing approximately 20,000 cells will be seeded on the artificial cornea and incubated at 37° C. in a 10% CO₂ incubator. After 2 hours, 2 ml of culture medium (DME-H16 supplemented with 15% fetal calf serum and 250 ng/ml of bFGF) will be added to the dish to totally submerge the polymer cornea and the cells. The human corneal endothelial cells initially with cover about 10% of the total surface are of the polymer cornea. The cells will be allowed to proliferate for 7 days, during which time the culture medium is chanced ever other day and bFGF at 250 ng/ml is added at the time of medium change. The cells will reach 100% confluence in 6-7 days at which time the artificial cornea will be ready for transplantation.

### EXAMPLE 7

### Coating the Polymer with a Deposit of Extracellular Matrix from Bovine Corneal Endothelial Cells for High Density Cell Seeding with Cultured Human Endothelial Cells

In another embodiment, the biopolymer is first molded into the shape of a cornea. Then a sample will be cut with an 11 mm trephine and place concave side up in a 35 mm tissue culture dish. Cultured bovine corneal endothelial cells will be detached from their culture dish and the subsequent cell suspension is adjusted to a density of 20,000 cells per ml. An aliquot of about 200 μl of the cell suspension will be added to the polymer cornea and the sample will be incubated at 37° C. on 10% CO₂ for 2 hours. Then about 2 ml of a culture medium containing DME-H16 supplemented with 10% calf serum, 5% fetal calf serum, 2% Dextran (40000 MV) and 50 ng/ml of bFGF will be added to the 25 mm dish to completely submerge the artificial cornea. The bovine endothelial cells will be allowed to proliferate for 7 days with bFGF at concentration of 50 ng/ml added to the medium every other day. At day-7 the culture medium will be removed and 2 ml of ammonium hydroxide (20 mM in distilled water) will be added and left for 5 minutes at 25° C. The artificial cornea is then washed 10 times with 2 ml of PBS per wash.

Cultured human corneal endothelial cell suspension prepared as previously described will be adjusted to final cell density of 100,000 cells/ml. An aliquot of 200 μl of the human cell suspension will be added to the artificial cornea with sufficient number of cells to cover over 95% of the surface area. A layer of 1% sodium hyaluronate (Healon® Advanced Medical Optics, Santa Ana, Calif.) of about 0.2-0.5 ml, is overlaid onto the cell layer to act as a protectant, and the artificial cornea will be incubated at 37° C. in 10% CO₂ for a period of 20 minutes to 24 hours. The polymer cornea is then ready to be transplanted.

### EXAMPLE 8

### Coating the Polymer with Extracellular Matrix Generated from Bovine Corneal Endothelial Cells for Sparse Cell Seeding with Cultured Human Corneal Endothelial Cells

The biopolymer cornea will be coated with extracellular matrix deposited by bovine endothelial cells as previously described in Example 7. The artificial cornea will be punched with an 11 mm trephine and placed concave side up in a 35 mm tissue culture dish. Cultured human corneal endothelial cells are prepared, as described above into a cell suspension. The final density of this cell suspension is adjusted to 20,000 cells per ml. An aliquot of 200 μl (containing 4000) cells will be added to the extracellular matrix coated polymer cornea. The sample will be allowed to incubate at 37° C. in 10% CO₂ during which time-culture medium will be changed every other day. At day 7 the human corneal endothelial cell will have proliferated to cover 100% of the surface are. The artificial cornea is then rinsed 3 times with PBS and is ready for transplantation.

### EXAMPLE 9

### Coating the Biopolymer with Diamond-Like Carbon (DLC) for High Density Seeding of Cultured Human Corneal Endothelial Cells

The biopolymer is molded into a cornea shape. The polymer cornea is then subjected to a process of carbon plasma deposit. The plasma equipment consists of a vacuum arc plasma gun [Lawrence Berkeley National Laboratory, Berkeley, Calif.] that is operated in repetitively pulsed mode so as to minimize high electrical power and thermal load concerns. Fitted with a carbon cathode, the plasma gun forms a dense plume of pure carbon plasma with a directed streaming energy of about 10 eV. The plasma is injected into a 90° magnetic filter (bent solenoid) so as to remove any particulate material from the cathode, and then transported through a large permanent magnet multipore configuration that serves to flatten the radial plasma profile; in this way the carbon plasma deposition is caused to be spatially homogenous over a large deposition area. To yet further enhance the film uniformity, the substrate(s) to be DLC coated are positioned on a slowly rotating disk, thus removing and azimuthal inhomogeneity. The plasma gun, vacuum chamber, and rotating disk assembly was used to form DLC films of about 20 to 4000 Å thick, preferably 200-400 Å thick. The plasma gun can be used to coat dishes, slides, blocks, beads, microcarriers, concave and convex surfaces artificial cornea, and polymer sheets.

After the DLC deposition, the artificial cornea will be punched with an 11 mm trephine and rinsed 3 times with PBS. A cultured human corneal endothelial cell suspension is prepared as described previously, with the final cell density adjusted to about 10⁶ cells per ml. An aliquot of 200 μl of cell suspension containing 200,000 cells will be added to the concave coated side of the artificial stroma with sufficient cells to cover over 95% of the surface area. The sample will be incubated at 37° C. in 10% CO₂ for a period of 20 minutes to 24 hours. The artificial cornea will be ready for transplantation.

### EXAMPLE 10

### Coating of a Biopolymer with Diamond-Like Carbon (DLC) for Seeding of Sparse Populations of Human Corneal Endothelial Cells

The biopolymer is molded into cornea shape and a carbon plasma (DLC) is deposited on the concave surface as described in Example 9. About a 200 μl aliquot of cultured human corneal endothelial cells with final concentration of 20,000 cells per ml will be added to the artificial stroma, which is placed inside a 35 mm tissue culture dish. The sample will be left for 2 hours at 37° C. in 10% CO₂. Then 2 ml of culture medium containing DME-H16 supplemented with 10% fetal calf serum and bFGF at 250 ng/ml will be added. The human corneal endothelial cells will be allowed to propagate for 7 days as described in Example 5. When the cells cover 100% of the surface are of the artificial cornea at day 7, the polymer cornea is rinsed three times with PBS and is ready for transplantation.

### EXAMPLE 11

### Artificial Full-Thickness Corneal Substitute Embedded with Attachment or Growth-Promoting Reagents and with Sparse Culture of Human Corneal Endothelial Cells Seeded onto the Concave Surface

In this embodiment, the biopolymer is embedded with or has incorporated into its composition during its synthesis an attachment mixture comprising of one or more of the following: fibronectin at concentrations ranging from 0.1 μg to 500 μg/ml of polymer gel, laminin at concentrations ranging from 0.1 μg to 500 μg/ml of polymer gel, RGDS at concentrations ranging from 0.1 μg to 100 μg/ml of polymer gel, bFGF conjugated with polycarbophil at concentrations ranging from 1 ng to 500 ng/ml of polymer gel, EGF conjugated with polycarbophil in concentrations ranging from 10 ng to 1000 ng/ml of polymer gel, and heparin sulfate at concentrations ranging from 1 μg to 500 μg/ml of polymer gel. The biopolymer is then molded into the desired shape of a cornea having a thickness equal to the thickness of a normal healthy human cornea about 0.4 to 0.8 mm, but can be thinner or thicker depending on the need.

Cultured human corneal endothelial cells at a density of between about 2000 to 2×10⁶ cells/ml, preferably about 20,000 cells/ml will be introduced to the concave surface of the corneal substitute. A sufficient volume of medium containing DME-H16 (supplemented with 15% fetal calf serum and 250 ng/ml of bFGF, and a cell density of 20,000 cells/ml) will be added to the concave side of the artificial stroma sitting inside a 25 mm culture dish. After about 2 hours of incubation at 37° C. and 10% CO₂, 2 ml of the same culture medium is added to the culture dish to fully submerge the cornea equivalent. The medium will be changed every other day and bFGF at 250 ng/ml is added after each medium change. At day 7 to day 10 the human corneal endothelial cells will attain a state of confluence on the artificial cornea. The artificial cornea will be rinsed three times with PBS and will be then ready for transplantation.

For treating a patient, the present invention will require the removal the damaged cornea from the recipient patient using known surgical techniques, implanting the artificial full thickness cornea, and securing said cornea by surgical or other means.

### EXAMPLE 12

### Artificial Half-Thickness Corneal Substitute Embedded with Attachment or Growth Promoting Reagents and with Sparse Culture of Corneal Endothelial Cells Seeded onto the Concave Surface

In this embodiment, the biopolymer is embedded with or has incorporated into its composition during its synthesis an attachment mixture detailed in Example 1. The biopolymer is the molded into the desired shape of a cornea, with a thickness up to half the thickness of a normal healthy human cornea about 0.4 to 0.8 mm, but can be thinner or thicker depending on the need. Cultured human corneal endothelial cells at a sparse density between about 2000 to 2×10⁶ cells/ml, preferably about 20,000 cells/ml, will be seeded onto the concave surface of the artificial stroma and the cells will be allowed to grow until they reach confluence at approximately seven to ten days. The half-thickness artificial cornea will be rinsed three times with PBS, and is then ready for transplantation.

The surgical procedure in this embodiment includes removing only the inner half of the recipient stroma that is associated with the damaged or diseased endothelium in a lamellar fashion, and then replacing it with the half-thickness artificial stroma with cultured human corneal epithelial cells grown on the concave side of it and secured by surgical or other means.

### EXAMPLE 13

### Artificial Full Thickness Corneal Substitutes Embedded with Attachment and/or Growth Promoting Reagents and with a Saturation, Density of Cultured Human Corneal Endothelial Cells Seeded onto the Concave Surface

In this embodiment the biopolymer is embedded with or has incorporated into its composition during its synthesis an attachment mixture detailed in Example 1. The biopolymer is then molded into the desired shape of a cornea with a thickness equal to that of a normal, healthy human cornea. A suspension of cultured human corneal endothelial cells at high density [10⁴ to 5×10⁶ cells/ml] (10⁶ cells/ml) will be prepared in culture medium containing DME-H16 supplemented with 1-5% fetal calf serum. The artificial corneal stroma will be punched with an 11 mm trephine. About a 200 μl aliquot of the cell suspension will be added to the concave side of the 11 mm diameter button. The sample will be incubated at 37° C. in 10% CO₂ for between 20 minutes to 24 hours. The artificial cornea will then be rinsed three times with PBS and is ready for transplantation.

When the corneal substrate is ready, the removal of the damaged cornea button from the recipient is accomplished by known surgical techniques, it will then be replaced with the artificial cornea, and secured by surgical or other means.

### EXAMPLE 14

### Artificial Half-Thickness Corneal Substitutes Embedded with Attachment and/or Growth Promoting Reagents and with a Saturation Density of Cultured Human Corneal Endothelial Cells Seeded onto the Concave Surface

In this alternate embodiment, the biopolymer is embedded with or has incorporated into its composition during its synthesis an attachment mixture detailed in Example 1. The biopolymer is then molded into the desired shape of a cornea with a thickness up to half that of a normal, healthy human cornea. A suspension of cultured human corneal endothelial cells at high density of between about 10⁴ to 5×10⁶ cells/ml, preferably 10⁶ cells/ml is used to seed a punched button of 11 mm diameter as described previously in Example 3. After the incubation period the corneal substitute is rinsed three times in PBS and is then ready for transplantation.

The surgical procedure includes removing from the patient only the inner half of the recipient stroma that is associated with the damaged or diseased endothelium in a lamellar fashion, and then replacing it with the half-thickness artificial stroma with cultured human corneal epithelial cells grown on the concave side of it and securing the new corneal implant by surgical or other means.

### EXAMPLE 15

### A Biopolymer Sheet of Uniform Thickness Between 10 and 100 microns as a Platform for Attachment of Cultured RPE Cells to be Delivered into the Sub-Retinal Space of the Eye for RPE Cell Transplantation

A thin sheet of biocompatible polymer of uniform thickness ranging from between about 1 to 1000 microns, preferably between about 10-100 microns, will be coated with cultured with RPE-cells. To achieve this step, cultured RPE cells of various animal species or human origin are detached from the culture dish with STV solution (0.05% trypsin, 0.02% EDTA in normal saline). The majority of the ST is removed as soon as the RPE cells round up but are still attached to the plate. The culture, with a thin film of STV still remaining, is then incubated at 37° C. in 10% CO₂ for 2-3 minutes. The RPE cells are removed by adding 5 ml of culture media (DME-H16 supplemented with 15% fetal calf serum) and washing gently with the aid of a 1 ml pipetman. The cell suspension is adjusted to a density of between about 2000 to 2×10⁶ cells/ml, preferably about 20,000 cells/ml. Sufficient quantity is added to the surface of the biopolymer sheet to form a meniscus inside a 25 mm culture dish. The sample is allowed to stand at 37° C. in 10% CO₂ for 2 hours. Then 2 ml of culture media (supplemented with 15% fetal calf serum and 100 ng/ml of bFGF) is added to the dish to totally submerge the polymer-sheet with RPE cells attached to it. The sheet can be allowed to float in the media but it is possible to attach it to the bottom with glue. The culture media will be changed every other day with the addition of bFGF at 100 ng/ml after each medium change. The RPE cells will become confluent in 7-10 days, which can be confirmed by observation on an inverted microscope. The sheet will then be cut into desired dimensions for covering the intended transplant are in the sub-retinal space. The sheet covered with RPE cells will be aspirated into a cannula. It will fold up with the RPE cells sitting on the top side of the sheet. After preparation of the implantation site as previously described, the sheet will be deposited onto the damaged area.

In another embodiment, the cultured RPE cells will be deposited at a high seeding density (2×10⁶ cells/ml) onto the polymer sheet ant the sample will be incubated at 37° C. in 10% CO₂ for 2 to 24 hours. The sheet will then, be washed extensively (3 to 5 times) in a large volume of BSS (10 ml each wash) to remove excessive cells not incorporated into the monolayer. The sheet is then cut into the desired dimensions and implanted as previously described.

### EXAMPLE 16

### Coating a Biodegradable Biopolymer with Uniform Thickness Between 10 and 100 microns with Cultured RPE Cells for Transplantation into the Sub-Retinal Space of the Eye

A biodegradable polymer sheet with uniform thickness Of 10-100 microns will be placed in a 35 mm culture dish. Cultured RPE cells are prepared in a suspension of cell density between about 2000 to 2×10⁶ cells/ml, preferably about 20,000 cells/ml as previously described. Sufficient volume of the cell suspension will be added to the sheet to form a meniscus. After about 2 hours of incubation at 37° C. in 10% CO₂2 ml of culture medium containing DME-H16 supplemented with 15% fetal calf serum and 100 ng/ml of bFGF will be added. The RPE layer will be allowed to grow to confluence as, previously described in Example 15 and RPE implantation is performed. Alternatively, the biodegradable polymer sheet can be deposited with a saturation density of cultures RPE cells left for 2 to 24 hours at 37° C. in 10% CO₂ environment. After the incubation it will be washed extensively (5-10′ times) with 10 ml of BSS and then implanted as previously described in Example 15.

### EXAMPLE 17

### Coating with Cultured RPE Cells a Biopolymer which is Embedded with, or has Incorporated into it During Synthesis, Attachment and Growth Promoting Agents for the Purpose of Transplantation into the Sub-Retinal Space of the Eye

In this embodiment, a biopolymer with uniform thickness of between about, 1 to 1000 microns, preferably between about 10 to 100 μm will be embedded with, or incorporated into it during synthesis, attachment and growth promoting agents comprising of one or more of the following: fibronectin at concentrations ranging from 1 μg to 200 μg/ml of polymer gel, laminin at concentrations ranging from 1 μg to 200 μg/ml of polymer gel, RGDS at concentrations ranging from 0.1 μg to 50 μg/ml of polymer, gel, bFGF conjugated with polycarbophil at concentrations ranging from 40 ng to 500 ng/ml of polymer gel, EGF conjugated with polycarbophil in concentrations ranging from 100 ng to 1000 ng/ml of polymer gel, and heparin sulfate at concentrations ranging from 0.1 μg to 100 g/ml of polymer gel. Then, as previously described, cultured RPE cells will be grown either at low seeding density (between about 10⁴ to 5×10⁵ cells/ml, preferably about 200,000 cells/ml) onto the polymer sheet for seven days as previously, or deposited at saturation density (about 2×10⁶ cells/ml) onto the polymer sheet. Then the implantation procedure will be executed as previously described in Example 15 for the RPE transplantation into the sub-retinal space of the eye.

### EXAMPLE 18

### Coating with Cultured RPE Cells a Biopolymer which is Embedded with, or has Incorporated into it During Synthesis, Attachment and Growth Promoting Agents for the Purpose of Transplantation into the Sub-Retinal Space of the Eye

In yet another embodiment contemplated, a biopolymer with uniform thickness of between about 1 to 1000 microns, preferably between about 10 to 100 μm will be embedded with, or incorporated into it during synthesis, attachment and growth promoting agents comprising of one or more of the following: fibronectin at concentrations ranging from 1 μg to 200 μg/ml of polymer gel, laminin at concentrations ranging from 1 μg to 200 μg/ml of polymer gel, RGDS at concentrations ranging from 0.1 μg to 50 μg/ml of polymer gel, bFGF conjugated with polycarbophil at concentrations ranging from 40 ng to 500 ng/ml off polymer gel, EGF conjugated with polycarbophil in concentrations ranging from 100 ng to 1000 ng/ml of polymer gel, and heparin sulfate at concentrations ranging from 0.1 μg to 100 μg/ml of polymer gel. Cultured RPE will be grown on the said biodegradable polymer sheet starting with a low seeding density between about 2000 to 2×10⁶ cells/ml, preferably about 20,000 cells/ml for seven days as previously mentioned in Example 15, or the RPE cells are deposited at saturation density (about 2×10⁶ cells/ml) onto the biodegradable polymer sheet also mentioned in Example 15. The implantation procedure will be carried out as previously described to accomplish the insertion of the RPE coated polymer sheet in the sub-retinal space of the eye.

Having described the invention, many modifications thereto will become apparent to those skilled in the art to which it pertains without deviation from the spirit of the invention as defined by the scope of the appended claims.

The disclosures of U.S. Patents, patent applications, and all other references cited above are all hereby incorporated by reference into this specification as if fully set forth in its entirety.

1. A method for modifying a biopolymer to enhance endothelial cell attachment and growth comprising coating a base biopolymer with an attachment mixture containing laminin, fibronectin, RGDS, bFGF conjugated with polycarbophil and EGF conjugated with polycarbophil for a period of time sufficient for corneal endothelial cells to attach to and grow on said biopolymer.

2. A method of making an artificial cornea comprising: a) a base biopolymer; b) molding the biopolymer into a desired shape; c) coating the biopolymer with an attachment mixture comprising laminin, fibronectin, RGDS, bFGF conjugated with polycarbophil and EGF conjugated with polycarbophil; d) incubating the reagent with the biopolymer at approximately 4° C. for a sufficient period of time to improve adherence of corneal endothelial cells; e) removing the attachment mixture; and f) seeding of corneal endothelial cells onto the biopolymer.

3. The method of claim 2 wherein the biopolymer is comprised of collagen IV.

4. The method of claim 2 wherein the seeding is at high density.

5. A method of making an artificial cornea comprising: a) a base biopolymer; b) molding the biopolymer into a desired shape; c) coating the biopolymer with a BCE-ECM coating comprising the steps of: 1) seeding onto the biopolymer at low density, a population of bovine corneal endothelial (BCE) cells in a culture media suitable for their growth; 2) allowing the BCE cells to grow to confluence; and 3) aspirating the media and treating the biopolymer with ammonium hydroxide for a sufficient period of time to remove the cells; d) washing the biopolymer with a suitable buffer; and e) seeding corneal endothelial cells onto the biopolymer and growing to confluence.

6. A method of making an artificial cornea comprising: a) a base biopolymer; molding the biopolymer into a desired shape; c) coating the biopolymer with Diamond-Like Carbon using a suitable process; d) washing the biopolymer with a suitable buffer; and e) seeding corneal endothelial cells onto the biopolymer and growing to confluence.

7. A method of growing endothelial cells suitable for use in a cornea comprising: a) a base biopolymer; b) molding the biopolymer into a desired shape; c) coating the biopolymer an adhesion factor mixture comprising a sufficient quantity of laminin, fibronectin, RGDS, and collagen IV in a suitable biological buffer; d) applying the biopolymer to the corneal button; and e) seeding corneal endothelial cells onto the biopolymer and growing to confluence.

8. A method of growing endothelial cells suitable for use in a cornea comprising: a) creating a base biopolymer in contact with an adhesion factor mixture comprising a sufficient quantity of laminin, fibronectin, RGDS, and collagen IV in a suitable biological buffer and a growth factor mixture comprising a sufficient quantity of bFGF, EGF and polycarbophil in a suitable biological buffer; b) molding the biopolymer into the shape of a cornea; c) applying the biopolymer to the corneal button; and d) seeding corneal endothelial cells onto the biopolymer and growing to confluence.

9. An attachment mixture comprising laminin, fibronectin, RGDS, bFGF conjugated with polycarbophil and EGF conjugated with polycarbophil in sufficient concentration to allow for growth of corneal endothelial cells in vitro.

10. An attachment mixture comprising: a) 10 μg to 500 μg/ml of fibronectin in PBS; b) 10 μg/ml to 500, μg/ml of laminin in PBS; c) 1 μg/ml to 100 μg/ml RGDS in PBS; d) 10 μg to 1000 μg of collagen type IV in 0.1 M acetic acid; e) 1 ng/ml to 500 ng/ml b-FGF in PBS; and f) 1 ng/ml to 500 ng/ml EGF in PBS.

11. An artificial full-thickness corneal transplant support comprising: a) a base biopolymer having a thickness of approximately an average cornea; b) incorporating into the biopolymer during its synthesis an attachment reagent comprising one or more of the following: laminin, fibronectin, RGDS, bFGF conjugated with polycarbophil, EGF conjugated with polycarbophil, and heparin sulfate; and c) molding the biopolymer into a desired shape of a cornea.

12. The composition of claim 11 wherein the biopolymer is comprised of collagen IV.

13. An artificial full-thickness corneal transplant comprising: a) a base biopolymer having a thickness of approximately an average cornea; b) incorporating into the biopolymer during its synthesis an attachment reagent comprising one or more of the following: laminin, fibronectin, RGDS, bFGF conjugated with polycarbophil, EGF conjugated with polycarbophil, and heparin sulfate; c) molding the biopolymer into the shape of a cornea; d) seeding HCEC onto the biopolymer and growing to confluence.

14. An artificial half-thickness corneal transplant support comprising: a) a base biopolymer having a thickness of approximately one half the thickness of an average cornea; b) incorporating into the biopolymer during its synthesis an attachment reagent comprising one or more of the following: laminin, fibronectin, RGDS, bFGF conjugated with polycarbophil, EGF conjugated with polycarbophil, and heparin sulfate; and c) molding the biopolymer into the shape of a cornea.

15. An artificial half-thickness corneal transplant comprising: a) a base biopolymer having a thickness of approximately one half the thickness of an average cornea; b) incorporating into the biopolymer during its synthesis an attachment reagent comprising one or more of the following: laminin, fibronectin, RGDS, bFGF conjugated with polycarbophil, EGF conjugated with polycarbophil, and heparin sulfate; c) molding the biopolymer into the shape of a cornea; d) seeding HCEC onto the biopolymer and growing to confluence.

16. The artificial cornea of claim 15 wherein the biopolymer is collagen IV.

17. The artificial cornea of claim 1 wherein the biopolymer is non-swelling in the presence of culture media.

18. A method of repairing a damaged cornea comprising the steps of: a) obtaining an artificial full-thickness cornea which has been seeded with HCEC and allowed to grow a sufficient period of time so that the HCEC are confluent; b) implanting the artificial full-thickness cornea of step a onto a damaged cornea; c) securing said cornea by surgical or other means.

19. A method of repairing a damaged cornea comprising the steps of: a) obtaining an artificial full-thickness cornea; b) overlaying said corneal surface with a biopolymer having confluent HCEC on it; c) implanting the artificial full-thickness cornea of step a onto a damaged cornea; d) securing said cornea by surgical or other means.

20. A method of repairing a damaged cornea comprising the steps of: a) obtaining an artificial half-thickness cornea which has been seeded with HCEC and allowed to grow a sufficient period of time so that the HCEC are confluent; b) implanting the artificial half-thickness cornea of step a onto a damaged cornea; c) securing said cornea by surgical or other means.

21. A method of repairing a damaged cornea comprising the steps of: a) obtaining an artificial half-thickness cornea; b) overlaying said corneal surface with a biopolymer having confluent HCEC on it; c) implanting the artificial half-thickness cornea of step a onto a damaged cornea; d) securing said cornea by surgical or other means.

22. A method for making retinal pigment epithelial (RPE) cells suitable for transplantation into a retina comprising the steps of: a) obtaining a biopolymer having a top and a bottom surface and having a thickness between about 10 to 100 m in thickness; b) placing said biopolymer in a medium suitable for the growth of RPE cells in vitro; c) seeding RPE cells onto the top surface of said biopolymer sheet at a certain density and allowing the RPE cells to grow to confluence; and d) removing said sheet and cutting to a desired size.

23. The method of claim 22 wherein the biopolymer is biodegradable.

24. The method of claim 22 wherein the biopolymer is embedded or has incorporated into it during its synthesis an attachment reagent, comprising one or more of the following: laminin, fibronectin, RGDS, bFGF conjugated with polycarbophil, EGF conjugated with polycarbophil, and heparin sulfate.

25. A composition comprising retinal pigment epithelial (RPE) cells suitable for transplantation into a retina made using the method of claim 22.

26. A method of repairing a retina in vivo comprising the steps of: a) identifying the damaged area of a retina to be repaired; b) aspirating remaining RPE cells from the damaged retinal area; c) obtaining retinal pigment epithelial (RPE) cells suitable for transplantation into a retina made by the method of claim 1; d) aspirating the biopolymer with the RPE on its top side into a cannula or other suitable aspiration means; e) injecting an air bubble of suitable size into the damaged area of a retina to be repaired; f) positioning the biopolymer with the RPE on its top side onto the damaged area with the cells on its top side; and g) aspirating the air bubble in the retinal space.
